(19) United States
(12) Patent Application Publication
(10) Pub. No.: US 2018/0100842 A1
(43) Pub. Date: Apr. 12, 2018

(54) Micro Sensor Package

(71) Applicant: Point Engineering Co., Ltd., Asan-si (KR)

(72) Inventors: Bum Mo Ahn, Suwon-si (KR); Seung Ho Park, Hwaseong-si (KR); Sung Hyun Byun, Hwaseong-si (KR)

(21) Appl. No.: 15/718,570

(22) Filed: Sep. 28, 2017

(30) Foreign Application Priority Data
Oct. 10, 2016 (KR) 10-2016-0130540

(51) Int. Cl. G01N 33/00; G01N 27/14

(52) U.S. Cl. CPC G01N 33/0027; G01N 27/14; G01N 33/0014

(57) ABSTRACT

A micro sensor package having a low thermal conductivity includes: a substrate on which a metal pattern is formed; a sensing chip disposed on the substrate; a cover covering the sensing chip and formed with a hole for supplying gas to the sensing chip; and a filter covering the hole, wherein the sensing chip comprises a sensor platform having a plurality of first pores formed along the up-down direction, and a sensor electrode formed on an upper portion or a lower portion of the sensor platform and electrically connected to the metal pattern.

### CROSS-REFERENCE TO RELATED PATENT APPLICATION

This U.S. non-provisional patent application claims priority under 35 U.S.C. § 119 of Korean Patent Application No. 10-2016-0130540 filed on Oct. 10, 2016 in the Korean Patent Office, the entire contents of which are hereby incorporated by reference.

### BACKGROUND

### 1. Technical Field

The present invention relates to a micro sensor package, more particularly, relates to a micro sensor package comprising a substrate on which a metal pattern is formed, a sensing chip disposed on the substrate, a cover covering the sensing chip and formed with a hole for supplying gas to the sensing chip, and a filter covering the hole, wherein the sensing chip comprises a sensor platform having a plurality of first pores formed along the up-down direction, and a sensor electrode formed on an upper portion or a lower portion of the sensor platform and electrically connected to the metal pattern.

### 2. Description of Related Art

A conventional miniature package for a gas sensor capable of sensing the amount of gas is shown in FIG. 1, which will be briefly described as follows.

A chip mounting portion 2 having a predetermined depth is formed at a central portion of a rectangular frame 1 made of an insulating material, and a sensor chip 4 is attached to the bottom surface of the chip mounting portion 2 with an epoxy 3.

A plurality of circuit lines 5 is formed inside of the frame 1, and a step portion 6 having a predetermined height along the inner circumferential surface is formed at the inner side edge of the chip mounting portion 2.

An inner terminal 5a extending from the one end of the circuit line 5 is formed on the step portion 6, and an outer terminal 5b extending from the other end (of the circuit line 5) is formed on the bottom edge of the frame 1.

A sensing film 16 for sensing gas is formed at the center portion of the upper surface of the sensor chip 4, and a plurality of sensor terminals 11 is formed at the edges for transmitting the resistance change detected by the sensing film 16 to the outside, and the sensor terminal 11 and the inner terminal 5a of the circuit line 5 are electrically connected by a silver paste 12, respectively.

A cap 13 is attached to the upper side of the frame 1 with an adhesive 14 so that the chip mounting portion 2 is covered, and in the cap 13, coupled in such a way, a plurality of gas holes 15 is formed so that gas can be introduced into the chip mounting portion 2.

In the micro-sized package for a gas sensor configured as described above, when a gas is introduced to inside the chip mounting portion 2 through the gas holes 15 in the cap 13, the resistance value of the sensing film 16 formed on the upper surface of the sensor chip 4 is varied due to the introduced gas, and the changing resistance values are transferred to a control unit (not shown) via the circuit lines 5, thereby measuring the amount of the gas.

Such a gas sensor is also provided with a heater, but the sensor chip 4 has a high thermal conductivity, so that there is a problem that a high power is required when the temperature needs to be raised to a high temperature.

### SUMMARY

### 1. Technical Problem

An objective of the present invention devised for solving the above described problems, is to provide a micro sensor package having a low thermal conductivity.

### 2. Solution to Problem

To achieve above described objective, a micro sensor package of the present invention is characterized in that and comprises: a substrate on which a metal pattern is formed; a sensing chip disposed on the substrate; a cover covering the sensing chip and formed with a hole for supplying gas to the sensing chip; and a filter covering the hole, wherein the sensing chip comprises: a sensor platform having a plurality of first pores formed along the up-down direction; and a sensor electrode formed on an upper portion or a lower portion of the sensor platform and electrically connected to the metal pattern.

To achieve above described objective, a micro sensor package of the present invention is characterized in that and comprises: a substrate on which a metal pattern is formed; a sensing chip disposed above the substrate; and a cover covering the sensing chip, wherein the sensing chip comprises: a sensor platform having a plurality of first pores formed along the up-down direction; and a sensor electrode formed on an upper portion or a lower portion of the sensor platform and electrically connected to the metal pattern, and wherein in the cover, a plurality of second pores for supplying gas to the sensing chip is penetratingly formed along the up-down direction.

The sensor platform may be an anodized film obtained by anodizing a base material made of a metal and then removing the base material.

The platform may be an anodized porous layer wherein the first pores are penetrating along the up-down direction.

The substrate may be a PCB.

The substrate may be formed of a ceramic material.

In the substrate, a plurality of third pores can be formed along the up-down direction.

The cover may be formed of a metallic material.

In the filter, a plurality of fourth pores may be penetratingly formed along the up-down direction, and the fourth pores may communicate with the hole.

The fourth pores may be formed by anodizing.

The filter may be subjected to hydrophobic treatment.

The filter may be installed outside of the cover.

The filter may be installed inside of the cover.

The filter may be surface treated so that a specific gas is selectively passing through.

The second pores may be formed by anodizing.

The cover may be subjected to hydrophobic treatment.

The cover may be surface treated so that a specific gas is selectively passing through.

The sensor platform may be formed with a resistor array electrically connected to the sensor electrode.

The resistor array may be formed on the same surface as the surface on which the sensor electrode is formed.

A resistor which is electrically connected to the sensor electrode is provided, and the resistor array may be formed on the substrate.

The sensor electrode and the metal pattern may be wire-bonded.

The first pores may be penetratingly formed along the up-down direction, and a first connecting portion for electrically connecting the sensor electrode and the metal pattern may be formed inside of at least a part of the first pores.

To achieve above described objective, a micro sensor package of the present invention is characterized in that and comprises: a substrate on which a metal pattern is formed; a sensing chip disposed on the substrate, wherein the sensing chip comprises a sensor platform and a sensing electrode formed on an upper portion or a lower portion of the sensor platform and electrically connected to the metal pattern, and wherein a plurality of third pores are penetratingly formed in the substrate along the up-down direction, and a second connecting portion electrically connected to the metal pattern may be formed inside of at least a part of the third pores.

The substrate may be an anodized porous layer.

To achieve above described objective, a micro sensor package of the present invention is characterized in that and comprises: a substrate on which a metal pattern is formed; a sensing chip disposed on the substrate, wherein the sensing chip comprises a sensor platform wherein a plurality of first pores is penetratingly formed along the up-down direction; a sensing electrode formed on an upper portion or a lower portion of the sensor platform and electrically connected to the metal pattern; and a first connecting portion for electrically connecting the metal pattern and the sensor electrode is formed inside of at least a part of the first pores.

To achieve above described objective, a micro sensor package of the present invention is characterized in that and comprises: a sensor platform wherein a plurality of first pores is formed along the up-down direction; a sensing chip comprising a sensor electrode formed in the sensor platform; and a cover that covers the sensor electrode, wherein at least a part of the plurality of first pores is penetrating through along the up-down direction, and wherein inside of the first pores that is being penetrated, a first connecting portion electrically connected to the sensor electrode is formed.

### BRIEF DESCRIPTION OF DRAWINGS

FIG. 1 is a view of a vertical cross-section of a miniature package for a gas sensor.

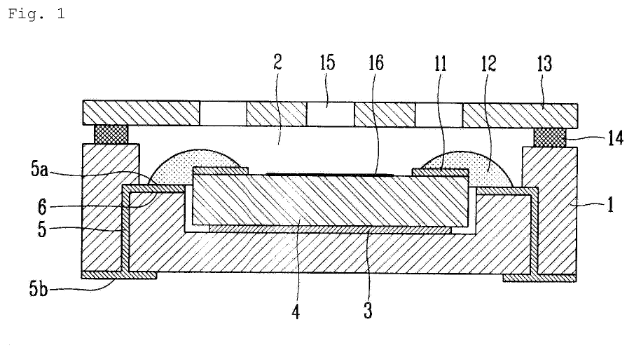

FIG. 2 is a cross-sectional view of a micro sensor package according to the first exemplary embodiment of the present invention.

FIG. 3 is a plan view of a sensing chip according to the first exemplary embodiment of the present invention.

FIG. 4 is an enlarged view of portion ‘A’ in FIG. 3.

FIG. 5 is a cross-sectional view along the line B-B in FIG. 3.

FIG. 6 is a cross-sectional view of a micro sensor package according to the second exemplary embodiment of the present invention.

FIG. 7 is a cross-sectional view of a micro sensor package according to the third exemplary embodiment of the present invention.

FIG. 8 is a cross-sectional view of a micro sensor package according to the fourth exemplary embodiment of the present invention.

FIG. 9 is a cross-sectional view of a micro sensor package according to the fifth exemplary embodiment of the present invention.

FIG. 10 is a cross-sectional view of a micro sensor package according to the sixth exemplary embodiment of the present invention.

FIG. 11 is a cross-sectional view of a micro sensor package according to the seventh exemplary embodiment of the present invention.

FIG. 12 is a cross-sectional view of a micro sensor package according to the eighth exemplary embodiment of the present invention.

FIG. 13 is a cross-sectional view of a micro sensor package according to the ninth exemplary embodiment of the present invention.

FIG. 14 is a cross-sectional view of a micro sensor package according to the tenth exemplary embodiment of the present invention.

FIG. 15 is a cross-sectional view of a micro sensor package according to the eleventh exemplary embodiment of the present invention.

FIG. 16 is a cross-sectional view of a micro sensor package according to the twelfth exemplary embodiment of the present invention.

### DETAILED DESCRIPTION OF EMBODIMENTS

Hereinafter, a preferred exemplary embodiment of the present invention will be described in detail with reference to the accompanying drawings as follows.

For reference, for the components of the present invention which will be described hereinafter and identical to those of the prior art, separate detailed descriptions will be omitted, but instead will be referred the prior art described above.

When it is mentioned that one part is on the “top” of other part, this means that the part may be directly on the top of the other part or another different part may be associated with therebetween. In contrast, if it is mentioned that one part is “directly on the top” of the other part, no other part is interposed therebetween.

The terminology used is merely to refer to a particular embodiment and are not intended to limit the present invention. As used herein, the singular forms also include the plural form of text that does not indicate clearly the significance of the opposite. The meaning of “comprising” as used in the specification embodies a specific characteristic, region, integers, steps, operations, elements and/or components, however, it does not exclude the presence or addition of other specific characteristics, regions, integers, steps, operations, elements, components and/or groups.

“Lower,” “upper,” and the like are the terms representing a relative space, and they may be used to describe the relationship of one part with respect to the other part illustrated in the drawing easier. These terms are intended to include other meanings or operations of the device that is used with the meaning intended in the drawing. For example, if the device in the drawing is flipped, the part which was in the “lower” side of the other part is now in the “upper” side of the other part. Thus, the exemplary term “lower” includes all of the upper and lower directions. Device may be rotated 90°, or may be rotated at a different angle, and also the terms indicating the relative space are interpreted accordingly.

### Embodiment 1

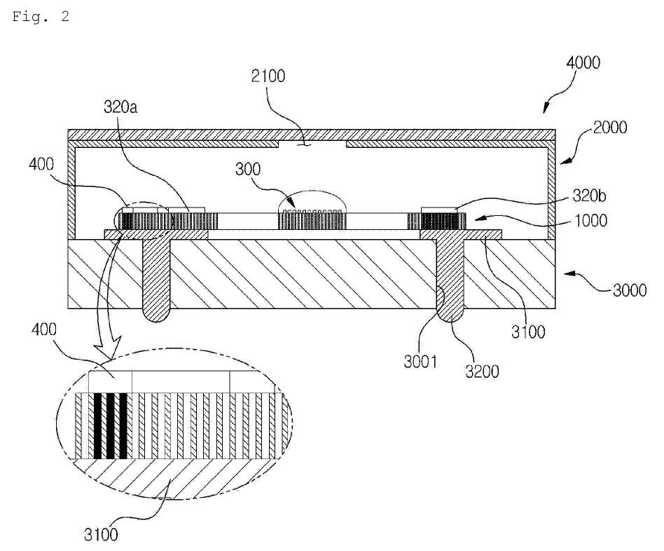

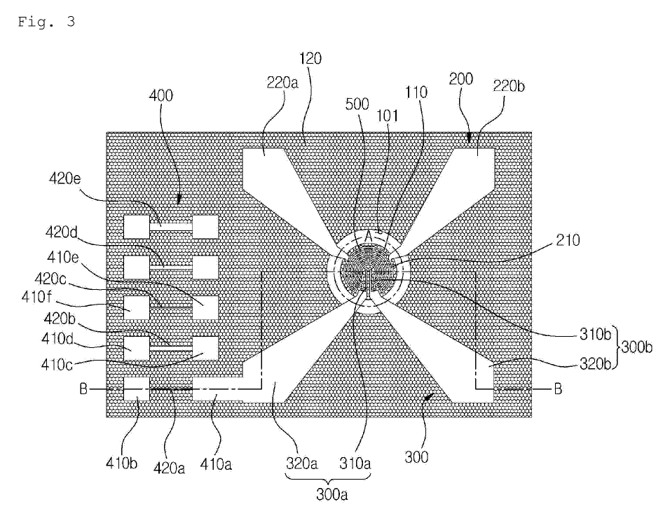

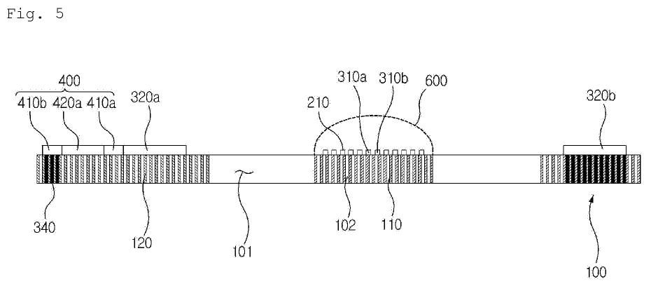

As illustrated in FIGS. 2 to 5, a micro sensor package of an exemplary embodiment is characterized in that and comprises: a substrate 3000 on which a metal pattern 3100 is formed; a sensing chip 1000 disposed above the substrate 3000; a cover 2000 covering the sensing chip 1000 and formed with a hole 2100 for supplying gas to the sensing chip 1000; and a filter 4000 covering the hole 2100, wherein the sensing chip 1000 comprises: a sensor platform 100 having a plurality of first pores 102 formed along the up-down direction; and a sensor electrode 300 formed on an upper portion or a lower portion of the sensor platform 100 and electrically connected to the metal pattern 3100.

The substrate 3000 whose upper and lower surfaces are formed in the shape of a flat plate is disposed horizontally.

The substrate 3000 is formed of an insulating material. Further, the substrate 3000 may be formed of a material having a low thermal conductivity.

Metal patterns 3100 are formed on both sides of the upper surface of the substrate 3000 so as to be spaced apart from each other. The metal pattern 3100 is horizontally formed along the left-to-right direction. A plurality of metal patterns 3100 are formed on the upper surface of the substrate 3000. On the metal pattern 3100, the sensing chip 1000 is mounted.

The substrate 3000 is made of PCB or ceramic material.

A through-hole 3001 is penetratingly formed in the substrate 3000 along the up-down direction at a position corresponding to the metal pattern 3100. A metal portion 3200 is disposed inside the through-hole 3001. The metal portion 3200 is filled in the through-hole 3001.

The upper portion of the metal portion 3200 is connected to the metal pattern 3100.

The metal portion 3200 is formed so as to be protruded below the lowermost end of the substrate 3000.

The micro sensor package is mounted on the PCB through the metal portion 3200.

The sensing chip 1000 is disposed above the substrate 3000 and mounted on the substrate 3000.

The sensing chip 1000 comprises: a sensor platform 100 wherein a plurality of first pores 102 is formed along the up-down direction; and a sensor electrode 300 formed on an upper portion or a lower portion of the sensor platform 100 and electrically connected to the metal pattern 3100.

The sensor platform 100 is formed of a porous material formed with a plurality of first pores 102 formed along the up-down direction, thereby improving the heat insulating property.

When an anodizing process is performed on a base material made of a metal, an anodized porous layer having a plurality of pores whose upper side is open is formed. The pores are formed in nanometer size. In here, the base material may be aluminum (Al), titanium (Ti), tungsten (W), zinc (Zn), or the like, but preferably it is made of aluminum or an aluminum alloy material which is lightweight, easy to process, excellent in thermal conductivity, and free from heavy metal contamination.

Further, when the barrier layer and the base material existing under the anodized porous layer are removed, the pores formed in the anodized porous layer are vertically penetrated.

The first pores 102 formed in the sensor platform 100 are formed by anodizing aluminum. Thus, the sensor platform 100 includes an anodized porous layer.

In addition, in the sensor platform 100, aluminum and the barrier layer are removed from the anodized aluminum oxide (AAO), and thereby the first pores 102 are penetrating along the up-down direction.

Unlike to the previous description, the sensor platform may be an anodized film obtained by removing only the base material after anodizing a base material made of a metal. That is, the sensor platform may be an anodized (oxide) film comprising an anodized porous layer and a barrier layer beneath the anodized porous layer.

The sensor platform 100 may be formed of a plate having a rectangular plan shape.

The sensor platform 100 comprises a first support 110 formed at the center of the sensor platform 100, a second support 120 spaced apart from the first support 110, and a bridge portion connecting the first support 110 and the second support 120.

The first support 110 is generally cylindrical in shape, and a plurality of the bridge portions is connected to the outer periphery thereof.

In the sensor platform 100, a plurality of air gaps 101 is formed in the vicinity of the first support 110, that is, between the first support 110 and the second support 120.

The air gap 101 is penetratingly formed along the up-down direction. That is, the air gap 101 is a space formed by penetrating through the sensor platform 100 from the upper surface to the lower surface.

The maximum width (left-to-right width) of the air gap 101 is formed to be wider than the maximum width of the first pore 102 and a sensor wiring or a heating wire 210 which will be described later. The air gap 101 is formed in the shape of an arc, and four of them are formed. A plurality of air gaps 101 is disposed spaced apart along the circumferential direction.

A plurality of air gaps 101 may be discontinuously formed. The air gap 101 and the bridge portion are alternately disposed around the periphery of the first support 110. Therefore, the first support 110 and the second support 120 are spaced apart from each other due to the air gap 101 at portions other than the bridge portion. The bridge portion is formed by discontinuously forming the air gap 101 by etching the vicinity of the first support 110. Thus, one end of the plurality of bridge portions is connected to the first support 110 and the other end is connected to the second support 120. The first support 110 and the second support 120 are connected to each other at four points by the four bridge portions.

The sensor electrode 300 is formed on the upper surface of the sensor platform 100.

The sensor electrode 300 detects a change in electrical characteristics when the gas is adsorbed to a sensing material 600.

The sensor electrode 300 comprises a first sensor electrode 300a and a second sensor electrode 300b disposed spaced apart from the first sensor electrode 300a. The first sensor electrode 300a and the second sensor electrode 300b are disposed spaced apart from each other and are formed symmetrically with respect to a center line disposed vertically on the plan surface.

Each of the first and second sensor electrodes 300a and 300b comprises the sensor wiring formed on the first supporting portion 110 and a sensor electrode pad formed on the bridge portion and the second support 120.

The first sensor electrode 300a comprises a first sensor wiring 310a formed on the upper surface of the first support 110 and a first sensor electrode pad 320a connected to the first sensor wiring 310a.

The second sensor electrode 300b comprises a second sensor wiring 310b formed on the upper surface of the first support 110 and a second sensor electrode pad 320b connected to the second sensor wiring 310b.

The sensor wiring comprises the first sensor wiring 310a and the second sensor wiring 310b. The sensor electrode pad comprises the first sensor electrode pad 320a and the second sensor electrode pad 320b. The width of the sensor wiring is formed to be constant. The sensor electrode pad is located on the upper surface of the bridge portion and the second support 120 and is formed to have a larger width than the first sensor wiring 310a and the second sensor wiring 310b. The sensor electrode pads of the first and second sensor electrodes 300a and 300b are formed to have a wider width as they travel towards the end portions thereof. That is, the sensor electrode pad is formed to have a narrower width as they travel towards the first sensor wiring 310a and the second sensor wiring 310b.

The sensor electrode 300 is formed of a mixture containing either one or at least one of Pt, W, Co, Ni, Au, and Cu.

A heater electrode 200 is formed on the upper surface of the sensor platform 100.

The upper sides of the first pores 102 located beneath the heater electrode 200 and the sensor electrode 300 are blocked by the heater electrode 200 and the sensor electrode 300 and the lower sides thereof are opened.

The heater electrode 200 comprises: a heating wire 210 formed on the first support 110 so as to be closer to the sensor wire than the sensor electrode pad; and the heater electrode pad connected to the heating wire 210 and formed on the second support 120 and the bridge portion

The heating wire 210 is formed on the upper surface of the first support 110 and is formed by surrounding at least a part of the first sensor wiring 310a and the second sensor wiring 310b from the outside thereof. The heater electrode pad comprises a first heater electrode pad 220a and a second heater electrode pad 220b which are connected to both ends of the heating wire 210 and are spaced apart from each other.

In the plan view, the heating wire 210 is formed to be symmetrical with respect to the vertical center line of the first support 110 and comprises a plurality of arc portions formed in the shape of an arc and a plurality of connecting portions connecting the arc portions.

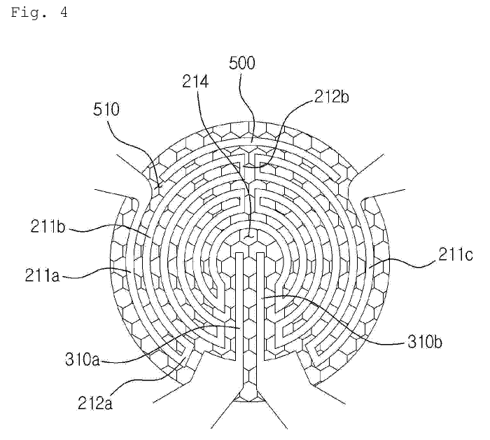

As illustrated in FIG. 4, the heating wire 210 is formed by repeatedly connecting a plurality of arc portions and connecting portions comprising: a first arc portion 211a adjacent to the air gap 101 and formed in the shape of an arc; a first bended portion 212a extending from the one end of the first arc portion 211a toward the inner side of the first support 110; a second arc portion 211b extending in the shape of an arc at an end of the first bended portion 212a and spaced apart from the first arc portion 211a; a second bended portion 212b extending from the end of the second arc portion 211b toward the inner side of the first supporting portion 110; a third arc portion 211c . . . , and so on.

The heating wire 210 is connected from the first arc portion 211a through the third arc portion 211c and forms an integral body.

Each of the plurality of arc portions of the heating wire 210 is formed in the shape of a substantially semicircle so as to form a circular shape as a whole. This improves the temperature uniformity of the first support 110 and the sensing material 600.

The center portion of the heating wire 210 is a point where both arc portions meet with each other, and the two arc portions in the shape of an arc join together to form a circular shape whose one side is open. And a separating space 214 is formed at the inner side thereof. The separating space 214 extends from the central portion of the first support 110 and the heating wire 210 up to the outermost sides of the first supporting portion 110 and the heating wire 210. The sensor wiring is disposed in the separating space 214. In addition, a first heater electrode pad 220a is connected to the other end of the first arc portion 211a and a second heater electrode pad 220b is connected to one end of the third arc portion 211c.

The heater electrode 200 is formed of a mixture containing either one or at least one of Pt, W, Co, Ni, Au, and Cu.

Meanwhile, between the end portions of the first arc portion 211a and the third arc portion 211c to which both ends of the heating wire 210, that is, the first heater electrode pad 220a and the second heater electrode pad 220b are connected, a dummy metal 500 is formed. The dummy metal 500 is formed on the upper surface of the first support 110.

The dummy metal 500 is disposed in the shape of an arc between the heating wire 210 of the heater electrode 200 and the air gap 101. The dummy metal 500 is formed spaced apart from the adjacent heating wire 210.

The dummy metal 500 is formed on the outer side of the heating wire 210 and is preferably a metal. The material of the dummy metal 500 may be the same as that of the electrode material, and the electrode material herein may be a metal such as platinum, aluminum, or copper.

The first arc portion 211a and the third arc portion 211c are formed to have a small central angle as compared with the remaining arc portions at the inner side thereof. A space 510 is formed between the end portions of the first arc portion 211a and the third arc portion 211c in the outer periphery of the heating wire 210, and the dummy metal 500 is located in the space 510.

The space 510 on the outer periphery of the heating wire 210 is partially filled as much as the formation area of the dummy metal 500. Therefore, when viewed in plan, since the outer periphery of the heating wire 210 and the dummy metal 500 form a substantially circular shape, the temperature uniformity of the first support 110 is improved, the temperature distribution of the heating wire 210 on the first support 110 heated with low power becomes more uniform.

The heater electrode pads comprise a first and second heater electrode pads 220a and 220b connected to both ends of the heating wire 210, respectively. In this way, the heater electrode pads are formed in at least two or more. The heater electrode pad is formed so as to have a wider width as it travels towards the outer side. That is, the heater electrode pad is formed to have a narrower width as it travels towards the heating wire 210. The heater electrode pad is formed to have a wider width than the heating wire 210.

The heater electrode pad and the sensor electrode pad are disposed radially with respect to the first support 110. The heater electrode pads and the sensor electrode pads are spaced apart from each other.

A protective layer (not shown) for preventing discoloration is formed on a portion of the upper side of the heater electrode 200 and the sensor electrode 300. The protective layer for preventing discoloration may be formed of an oxide-based material. Further, the protective layer for preventing discoloration is formed of at least one of tantalum oxide (TaOx), titanium oxide (TiO₂), silicon oxide (SiO₂), and aluminum oxide (Al₂O₃).

The heating wire 210 and the first and second sensor wirings 310a and 310b are surrounded by the air gap 101. The air gap 101 is disposed around the heating wire 210 and the first and second sensor wirings 310a and 310b. The air gap 101 is disposed at the side of the heating wire 210 and the first and second sensor wirings 310a and 310b.

More specifically, the air gap 101 is formed between the first sensor electrode pad 320a and the first heater electrode pad 220a of the first sensor electrode 300a and between the first heater electrode pad 220a and the second heater electrode pad 220b and between the second heater electrode pad 220b and the second sensor electrode pad 320b of the second sensor electrode 300b and between the second sensor electrode pad 320b of the second sensor electrode 300b and the first sensor electrode pad 320a of the first sensor electrode 300a. That is, the air gap 101 is formed in a region excluding the portion supporting the heater electrode 200 and the sensor electrode 300.

Due to the air gap 101, the first support 110 which commonly supports the heating wire 210 and the sensor wiring, the second support 120 which supports the heater electrode pad and the sensor electrode pad, and the bridge portion are formed in the sensor platform 100.

The first support 110 is formed to have a larger area than the heating wire 210 and the sensor wiring.

The first support 110 is formed with a heating wire 210 and a sensing material 600 covering the sensor wiring. That is, the sensing material 600 is formed at a position corresponding to the first support 110. The sensing material 600 is formed by printing. In this way, once the sensing material 600 is formed by printing, a trace resembling a mesh network is left on the surface of the sensing material 600 after the sensing material 600 is formed.

Further, a resistor array 400 electrically connected to the sensor electrode pad of the sensor electrode 300 is formed on the sensor platform 100.

The resistor array 400 is formed on the upper surface of the sensor platform 100 and formed on the same plane as the sensor electrode 300.

The resistor array 400 is disposed spaced apart from the heater electrode 200.

The resistor array 400 is disposed at the second support 120.

In the present exemplary embodiment, the gas sensing portion (sensor electrode and heater electrode) is disposed at the right side of the sensor platform 100, and the resistor array 400 is disposed at the left side of the sensor platform 100. Accordingly, the air gap 101 is disposed between the resistor array 400 and the first support 110.

The resistor array 400 includes at least two resistors.

At least one of the resistors is formed in the shape of a sheet resistor or a fine pattern (line shape), so that the volume of the resistor array 400 can be minimized.

More specifically, the resistor array 400 comprises first, second, third, fourth and fifth resistor pads 410a, 410b, 410c, 410d and 410e and first, second and third resistors 420a, 420b and 420c.

Each of the resistor pads is disposed to be spaced apart from each other.

The first resistor pad 410a is connected to the first sensor electrode pad 320a of the sensor electrode 300. Unlike to the previous description, the first resistor pad may be connected to the second sensor electrode pad of the sensor electrode 300.

The first resistor pad 410a of the resistor array 400 connected to the sensor electrode 300 is integrally formed with the first sensor electrode pad 320a or the second sensor electrode pad 320b of the sensor electrode 300. Therefore, the resistor array 400 is formed integrally with the sensor electrode 300. Unlike to this, the resistor array and the sensor electrode may be formed separately.

The first resistor pad 410a is connected to at least one other resistor pad via at least one resistor.

The first resistor pad 410a is connected to one side of the first resistor 420a and the second resistor pad 410b is connected to the other side of the first resistor 420a.

The third resistor pad 410c is connected to one side of the second resistor 420b and the fourth resistor pad 410d is connected to the other side of the second resistor 420b.

A fifth resistor pad 410e is connected to one side of the third resistor 420c and the sixth resistor pad 410f is connected to the other side of the third resistor 420c.

In the present exemplary embodiment, the resistor array may comprise five resistors. The resistor array comprises first, second, third, fourth and fifth resistors 420a, 420b, 420c, 420d and 420e.

Each of the resistors in the present exemplary embodiment has resistive pads on both sides. Therefore, the resistance pads are connected to one side and the other side of the fourth and fifth resistors 420d and 420e, which are the remaining resistors.

Each of the five resistors may have a different resistance value, or at least two of the five resistors have different resistance values.

The first resistor 420a connected to the first sensor electrode pad 320a of the sensor electrode 300 may have the largest value among the five resistors. The first, second, third, fourth and fifth resistors 420a, 420b, 420c, 420d and 420e are provided as sheet resistors, and the resistance becomes larger as the line width (front-to-rear width) becomes thinner.

The resistor array 400 is connected only to the first sensor electrode pad 320a of the sensor electrode 300 and the sensor electrode 300 is connected to the resistor array 400 in series.

Each of the resistor pads may be selectively connected to the sensor electrode 300 through wire bonding or the like depending on the resistance of the sensing material 600.

At least two of the resistors may be connected in series or in parallel.

The first pores 102 beneath the sensor electrode pads or the resistor pads or the heater electrode pads are penetratingly formed along the up-down direction. The first pores 102 disposed between the sensor electrode pad or the resistor pad or the heater electrode pad and a metal pattern 3100 is penetratingly formed along the up-down direction.

That is, the first pores 102 are formed so as to penetrate from the surface, wherein the sensor electrode pads or the resistor pads or the heater electrode pads are formed, into the opposite surface.

Inside the plurality of first pores 102 beneath the sensor electrode pad or the resistance pad or the heater electrode pad, a first connecting portion 340 is formed for electrically connecting a heater electrode pad of the sensor electrode 300 or a heater electrode pad of the heater electrode 200 to a metal pattern 3100 disposed at the opposite side of the sensor electrode 300 and the heater electrode 200. That is, the first connecting portion 340 is filled in the first pores 102. The lower part of the first connecting portion 340 may be formed so as to be protruded lower than the lower surface of the sensor platform 100.

The first connecting portion 340 serves as a medium for connecting a metal or a pattern or an electrode disposed on the opposite side of the sensor electrode 300.

The connection means a direct connection or an indirect connection. Unlike to the above description, in the sensor platform, a sensor bonding portion connected to a lower portion of the first connecting portion may be further formed on a bottom surface which is opposite to the surface on which the sensor electrode, the heater electrode, and the resistor are formed. The upper portion of the sensor bonding portion is horizontally formed along the left-to-right direction so as to be connected to the plurality of first connecting portions. A metal pattern may be connected to a lower portion of the sensor bonding portion. That is, the first connecting portions and the metal pattern may be indirectly connected through the sensor bonding portion.

The first connecting portion 340 is formed in the shape of a column having a diameter of several nanometers.

In the present exemplary embodiment, since the resistor array 400 is integrally formed on the first sensor electrode pad 320a of the sensor electrode 300, at least one of the five resistor pads 410b, 410c, 410d, and 410e or the second sensor electrode pad 320b, and the first and second heater electrode pads 220a and 220b are connected to the upper portion of the first connecting portion 340.

In this way, the first connecting portion 340 is formed inside the first pores 102 so that the first connecting portion 340 can be formed without any additional etching operation and may be mounted in the form of a surface mount device (SMD) without wire bonding.

Unlike to the previous description, the substrate may also be formed to include an anodized porous layer in which a plurality of third pores is formed along the up-down direction, such as the sensor platform 100 as previously described.

The third pores of the substrate may be penetratingly formed along the up-down direction. In this case, the PCB to which the metal pattern and the micro sensor package are mounted may be electrically connected by a second connecting portion disposed inside the third pores. The second connecting portion is filled in the third pores, the upper portion of the second connecting portion is connected to the metal pattern, and the lower portion is connected to the PCB where the micro sensor package is mounted. Furthermore, a substrate bonding portion may be formed in the lower surface of the substrate along the left-to-right direction so as to be disposed between the lower portion of the second connecting portion and the PCB on which the micro sensor package is mounted. And the substrate bonding portion is connected to a lower portion of the second connecting portion.

A cover 2000 covers a sensing chip 1000 comprising the sensor electrode 300, and a hole 2100 for supplying a gas to the sensing chip 1000 is formed.

The cover 2000 is formed of a metal material such as stainless steel (SUS).

The cover 2000 is installed on the upper surface of the substrate 3000 through an adhesive or the like. The lower end of the cover 2000 is attached along the edge of the substrate 3000.

In the cover 2000, a cavity wherein the sensing chip 1000 and the metal pattern 3100 are disposed is formed. The cavity is formed so that the lower portion thereof is open. The cover 2000 surrounds the top and sides of the sensing chip 1000 and the metal pattern 3100.

Meanwhile, the cover 2000 is formed of material having the same or similar shrinkage rate or expansion rate of the substrate 3000, so that the manufacturing becomes easier and the cover 2000 and the substrate 3000 can be prevented from being separated even if they are contracted or expanded.

In the cover 2000, a hole 2100 is penetratingly formed along the up-down direction. The hole 2100 communicates with the cavity.

The hole 2100 is disposed to correspond to the position of the sensing material 600.

The filter 4000 is provided so as to cover the hole 2100.

Therefore, the gas is supplied to the sensing chip 1000 after passing through the filter 4000.

The filter 4000 is formed in the shape of a plate and installed by attaching to the upper surface of the upper plate of the cover 2000 using an adhesive or the like. Therefore, the filter 4000 is installed outside the cover 2000.

The filter 4000 may be formed of a porous material.

Further, the filter 4000 may be formed of an anodized aluminum porous layer wherein a plurality of fourth pores is penetratingly formed along the up-down direction through anodizing process. The fourth pores communicate with the hole 2100.

The inside of the fourth pore of the filter 4000 is subjected to a hydrophobic surface treatment to prevent moisture from infiltrating into the gas detecting portion.

The filter 4000 may be surface treated such that a specific gas is selectively passing through or being blocked. Unlike to this, the filter 4000 may have a different diameter of the fourth pore for selective passing of gas. That is, the diameter of the fourth pore may be different depending on the type of gas to be detected.

Hereinafter, the operation of the present exemplary embodiments having the above described configuration will be described.

In order to measure the gas concentration, a constant electric power is first applied to the two heater electrode pads of the heater electrode 200 to heat the sensing material 600 to a constant temperature.

In the heated sensing material 600, the gas inside the cavity that has been passed through the filter 4000 is adsorbed or desorbed.

As a result, the electrical conductivity between the first sensor wiring 310a and the second sensor wiring 310b changes, and the sensing signal is amplified through the resistor array 400 to detect the gas.

Further, in order to perform more precise measurement, other gas species or moisture already adsorbed to the sensing material 600 are heated at a high temperature by the heater electrode 200 so as to be forcibly removed from the sensing material 600, and thereby, the sensing material 600 is recovered to its initial state so that the gas concentration is measured.

### Embodiment 2

In describing the micro sensor package according to the second exemplary embodiment of the present invention, same symbols will be used for the same or similar elements as those of the micro sensor package according to the first exemplary embodiment of the present invention, and the detailed description and illustration will be omitted.

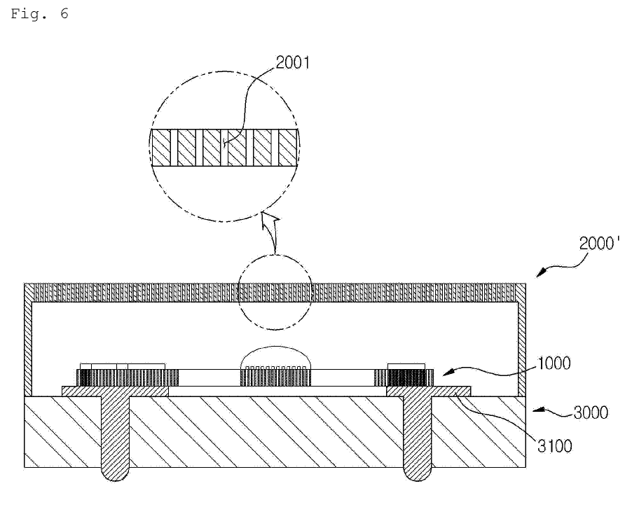

As illustrated in FIG. 6, a micro sensor package according to the second exemplary embodiment characterized in that and comprises: a substrate 3000 on which a metal pattern 3100 is formed; a sensing chip 1000 disposed above the substrate 3000; and a cover 2000′ covering the sensing chip 1000, wherein the sensing chip 1000 comprises: a sensor platform having a plurality of first pores formed along the up-down direction; and a sensor electrode formed on an upper portion or a lower portion of the sensor platform and electrically connected to the metal pattern 3100, and wherein in the cover 2000′, a plurality of second pores 2001 for supplying gas to the sensing chip 1000 is penetratingly formed along the up-down direction.

Since the substrate 3000 and the sensing chip 1000 are the same as those of the first exemplary embodiment, the detailed description thereof will be omitted.

The cover 2000′ is installed on the upper surface of the substrate 3000 using an adhesive or the like. The lower end of the cover 2000′ is installed along the edge of the substrate 3000.

A cavity in which the sensing chip 1000 and the metal pattern 3100 are disposed is formed in the cover 2000′. The cavity is formed so that the lower portion thereof is open. The cover 2000′ surrounds the top and sides of the sensing chip 1000 and the metal pattern 3100.

In a part or all of the cover 2000′, a plurality of second pores 2001 is formed along the up-down direction for supplying gas to the sensing chip 1000. That is, the cover 2000′ is provided with a porous layer. The second pores 2001 communicate with the cavity. The second pore 2001 has a diameter of nanometer size.

In this way, the second pores 2001 are formed in the cover 2000′ so that the cover 2000′ can also function as a filter simultaneously.

When the second pores 2001 are formed only in a portion of the cover 2000′, the porous layer is formed on the top plate of the cover 2000′. More specifically, the porous layer is disposed to correspond to the position of the sensing material.

The second pores 2001 are formed by anodizing aluminum.

A hydrophobic surface treatment may be performed inside the second pores 2001 of the cover 2000′.

Further, the cover 2000′ may be surface-treated so that a specific gas is selectively passing through. The diameter of the second pores 2001 of the cover 2000′ may be different depending on the types of gas to be detected.

In the sensing chip 1000, a resistor array is formed on the upper surface of the sensor platform. The resistor array is integrally formed with the sensor electrode. The resistor array and the metal pattern 3100 are connected through a first connecting portion filled in the first pores of the sensor platform.

### Embodiment 3

In describing the micro sensor package according to the third exemplary embodiment of the present invention, same symbols will be used for the same or similar elements as those of the micro sensor package according to the first and second exemplary embodiments of the present invention, and the detailed description and illustration will be omitted.

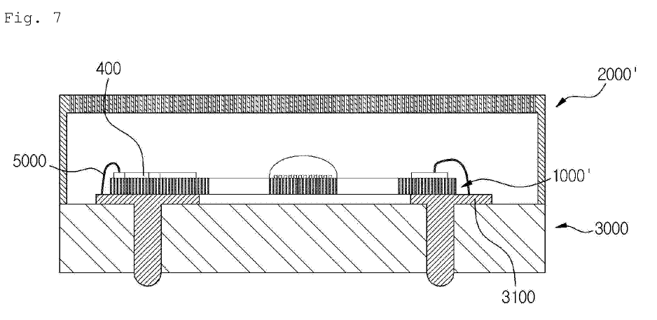

As illustrated in FIG. 7, in a micro sensor package according to the third exemplary embodiment, a first connecting portion is not formed in a sensing chip 1000′, and the sensor electrode of the sensing chip 1000′ and a metal pattern 3100 formed on the upper surface of a substrate 3000 and are wire-bonded through a wire 5000.

As in the first embodiment, the sensing chip 1000′ has a resistor array 400 formed on the upper surface of the sensor platform. The resistor array 400 is integrally formed on the first sensor electrode pad of the sensor electrode.

Thus, one end of the wire 5000 connected to the first sensor electrode pad is connected to the resistance pad of the resistor array 400 and the other end is connected to the metal pattern 3100. Accordingly, the first sensor electrode pad is connected to the metal pattern 3100 by the wire 5000 through the resistor array 400.

One end of the remaining wire 5000 is connected to the second sensor electrode pad and the first and second heater electrode pads, respectively, and the other end is connected to the metal pattern 3100.

The wire 5000 is disposed inside a cover 2000′. The cover 2000′ also has an anodized porous layer as in the second exemplary embodiment, and functions as a filter simultaneously.

### Embodiment 4

In describing the micro sensor package according to the fourth exemplary embodiment of the present invention, same symbols will be used for the same or similar elements as those of the micro sensor package according to the first, second, and third exemplary embodiments of the present invention, and the detailed description and illustration will be omitted.

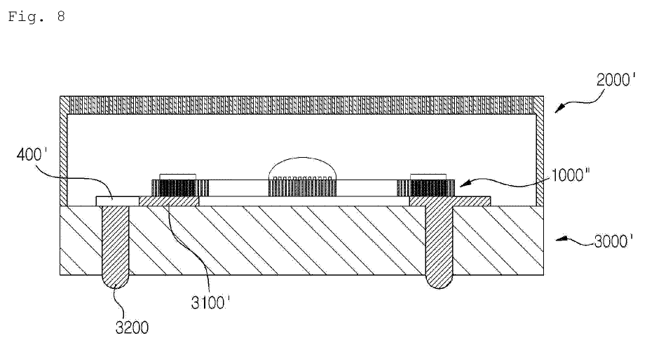

As illustrated in FIG. 8, in the micro sensor package according to the fourth embodiment, a resistor array 400′ electrically connected to the sensor electrode of a sensing chip 1000″ is formed on a substrate 3000′.

The resistor array 400′ is formed on the upper surface of the substrate 3000′ so as to be disposed outside the sensing chip 1000″. The resistor array 400′ may be formed integrally with a metal pattern 3100′.

A heater electrode pad is connected to an upper portion of the first connecting portion of the sensing chip 1000″, and a metal pattern 3100′ is connected to a lower portion of the first connecting portion.

The metal pattern 3100′, connected to one of the sensor electrode pads (first sensor electrode pad), is connected to one side of the resistor array 400′. The upper part of a metal portion 3200 is connected to the other side of the resistor array 400′. And the remaining metal pattern 3100′ is directly connected to the metal portion 3200. The lower part of the first connecting portion may be directly connected to one side of the resistor array 400′ without passing through the metal pattern 3100′.

The resistor array 400′ is disposed inside a cover 2000′.

The cover 2000′ also has an anodized porous layer as in the second exemplary embodiment, and functions as a filter simultaneously.

### Embodiment 5

In describing the micro sensor package according to the fifth exemplary embodiment of the present invention, same symbols will be used for the same or similar elements as those of the micro sensor package according to the first, second, third, and fourth exemplary embodiments of the present invention, and the detailed description and illustration will be omitted.

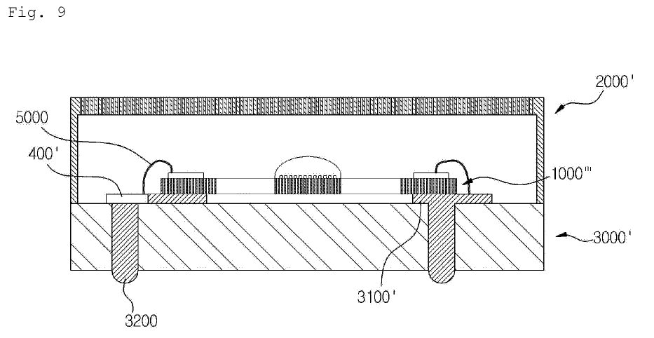

As illustrated in FIG. 9, in the micro sensor package according to the fifth exemplary embodiment, a first connecting portion is not formed in a sensing chip 1000″, and a sensor electrode of a sensing chip 1000″ and a metal pattern 3100′ formed on the upper surface of a substrate 3000′ are connected through a wire 5000, and a resistor array 400′ is formed on the substrate 3000′.

A second sensor electrode pad and a heater electrode pad of the sensing chip 1000″ are connected to the metal pattern 3100′ through the wire 5000. A metal portion 3200 is connected to the metal pattern 3100′.

The first sensor electrode pad is connected to the one side of the resistor array 400′ through the wire 5000.

The upper portion of the metal portion 3200 is connected to the other end of the resistor array 400′.

In this way, when the resistor 400′ is formed on the substrate 3000′, a part of the sensor electrode is connected to the metal portion 3200 through the wire 5000 and the resistor array 400′. The rest of the sensor electrode and the heater electrode are connected to the metal portion 3200 through the wire 5000 and the metal pattern 3100′.

The wire 5000 and the resistor array 400′ are disposed inside a cover 2000′.

The cover 2000′ also has an anodized porous layer as in the second exemplary embodiment, and functions as a filter simultaneously.

### Embodiment 6

In describing the micro sensor package according to the sixth exemplary embodiment of the present invention, same symbols will be used for the same or similar elements as those of the micro sensor package according to the first, second, third, fourth, and fifth exemplary embodiments of the present invention, and the detailed description and illustration will be omitted.

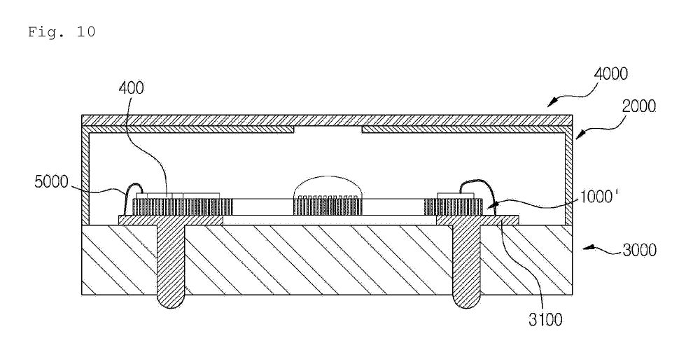

As illustrated in FIG. 10, in the micro sensor package according to the sixth exemplary embodiment, a first connecting portion is not formed in a sensing chip 1000′, a sensor electrode of the sensing chip 1000′ and a metal pattern 3100 formed on the upper surface of a substrate 3000 is wire-bonded through a wire 5000.

As in the first exemplary embodiment, the sensing chip 1000′ has a resistor array 400 formed on the upper surface of the sensor platform. The resistor array 400 is integrally formed on the first sensor electrode pad of the sensor electrode.

Thus, one end of the wire 5000 is connected to the resistor pad of the resistor array 400, and the other end is connected to the metal pattern 3100.

The wire 5000 is disposed inside a cover 2000. As in the first exemplary embodiment, a filter 4000 is attached on the cover 2000.

### Embodiment 7

In describing the micro sensor package according to the seventh exemplary embodiment of the present invention, same symbols will be used for the same or similar elements as those of the micro sensor package according to the first, second, third, fourth, fifth, and sixth exemplary embodiments of the present invention, and the detailed description and illustration will be omitted.

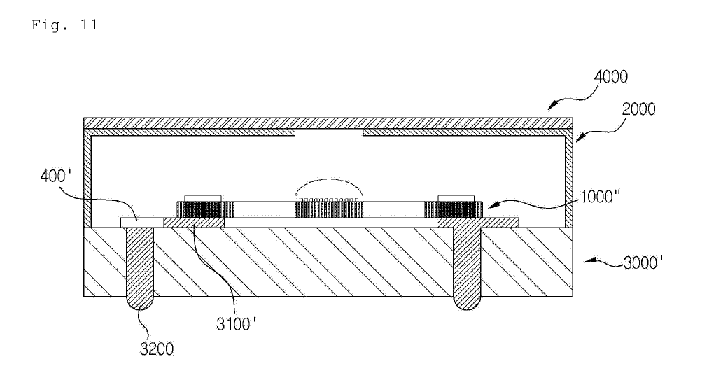

As illustrated in FIG. 11, in the micro sensor package according to the seventh exemplary embodiment, a resistor array 400′ electrically connected to a sensor electrode of a sensing chip 1000″ is formed on a substrate 3000′.

The resistor array 400′ is formed on the upper surface of the substrate 3000′ so as to be disposed outside the sensing chip 1000″.

A sensor electrode pad and a heater electrode pad are connected to an upper portion of the first connecting portion of the sensing chip 1000″, and a metal pattern 3100′ is connected to the lower portion of the first connecting portion.

The metal pattern 3100′, connected to one of the sensor electrode pads, is connected to one side of the resistor array 400′. The upper part of a metal portion 3200 is connected to the other side of the resistor array 400′. And the remaining metal pattern 3100′ is directly connected to the metal portion 3200.

The resistor array 400′ is disposed inside a cover 2000. As in the first exemplary embodiment, a filter 4000 is attached on the cover 2000.

### Embodiment 8

In describing the micro sensor package according to the eighth exemplary embodiment of the present invention, same symbols will be used for the same or similar elements as those of the micro sensor package according to the first, second, third, fourth, fifth, sixth, and seventh exemplary embodiments of the present invention, and the detailed description and illustration will be omitted.

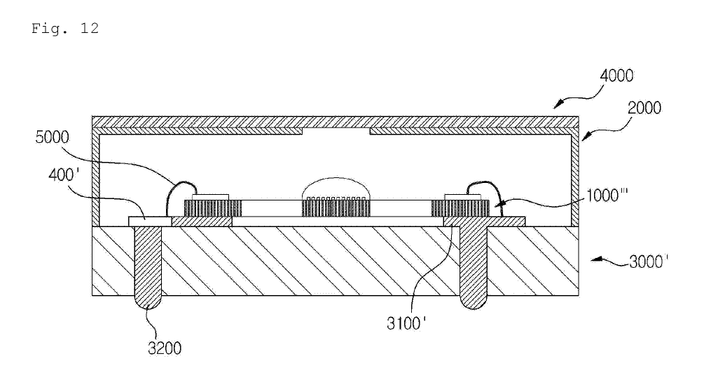

As illustrated in FIG. 12, in the micro sensor package according to the seventh exemplary embodiment, A sensor electrode of a sensing chip 1000″ and a metal pattern 3100′ formed on the upper surface of a substrate 3000′ are connected via a wire 5000, and a resistor array 400′ is formed on the substrate 3000′.

The second sensor electrode pad and the heater electrode pad of the sensing chip 1000″ are connected to the metal pattern 3100′ through the wire 5000. A metal portion 3200 is connected to the metal pattern 3100′.

The first sensor electrode pad is connected to one side of the resistor array 400′ through the wire 5000.

The upper portion of a metal portion 3200 is connected to the other side of the resistor array 400′.

The wire 5000 and the resistor array 400′ are disposed inside a cover 2000. As in the first exemplary embodiment, a filter 4000 is attached on the cover 2000.

### Embodiment 9

In describing the micro sensor package according to the ninth exemplary embodiment of the present invention, same symbols will be used for the same or similar elements as those of the micro sensor package according to the first, second, third, fourth, fifth, sixth, seventh, and eighth exemplary embodiments of the present invention, and the detailed description and illustration will be omitted.

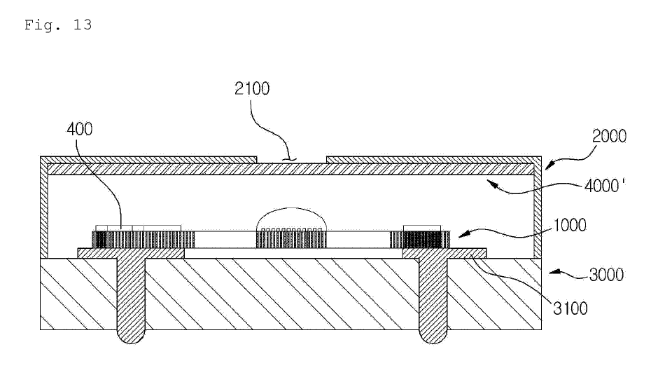

As illustrated in FIG. 13, in a micro sensor package according to the ninth exemplary embodiment, a filter 4000′ is formed inside a cover 2000.

The filter 4000′ is attached to the lower surface of the upper plate of the cover 2000. The gas introduced through a hole 2100 flows into the cavity of the cover 2000 after passing through the filter 4000′.

The material of the filter 4000′ may be the same as that of the filter of the first exemplary embodiment.

In a sensing chip 1000, a resistor array 400 is formed on the upper surface of a sensor platform. The resistor array 400 is integrally formed on the sensor electrode. The resistor array 400 and the metal pattern 3100 of a substrate 3000 are connected through a first connecting portion filled in first pores of the sensor platform.

### Embodiment 10

In describing the micro sensor package according to the tenth exemplary embodiment of the present invention, same symbols will be used for the same or similar elements as those of the micro sensor package according to the first, second, third, fourth, fifth, sixth, seventh, eighth, and ninth exemplary embodiments of the present invention, and the detailed description and illustration will be omitted.

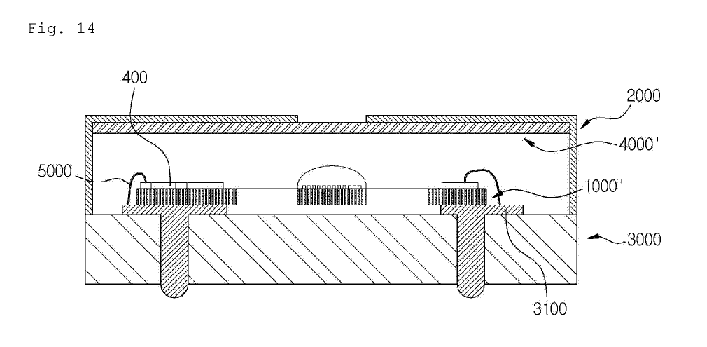

As illustrated in FIG. 14, in a micro sensor package according to the tenth exemplary embodiment, a sensor electrode of a sensing chip 1000′ and a metal pattern 3100 formed on the upper surface a substrate 3000 are wire-bonded through a wire 5000.

As in the first exemplary embodiment, the sensing chip 1000′ has a resistor array 400 formed on the upper surface of the sensor platform. The resistor array 400 is integrally formed on a first sensor electrode pad of the sensor electrode.

Thus, one end of the wire 5000 is connected to the resistance pad of the resistor array 400, and the other end is connected to a metal pattern 3100.

The wire 5000 is disposed inside a cover 2000. A filter 4000′ is attached on the cover 2000.

### Embodiment 11

In describing the micro sensor package according to the eleventh exemplary embodiment of the present invention, same symbols will be used for the same or similar elements as those of the micro sensor package according to the first, second, third, fourth, fifth, sixth, seventh, eighth, ninth, and tenth exemplary embodiments of the present invention, and the detailed description and illustration will be omitted.

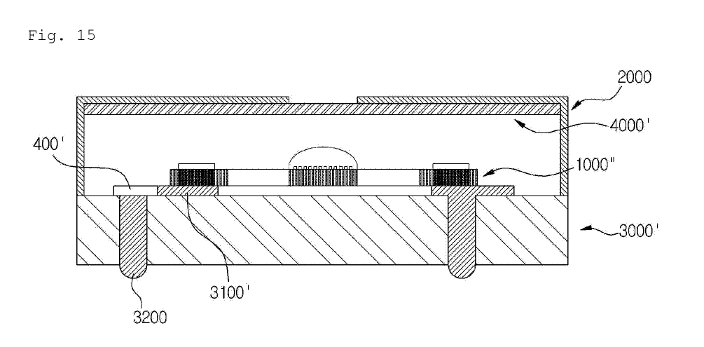

As illustrated in FIG. 15, in a micro sensor package according to the eleventh exemplary embodiment, a resistor array 400′ electrically connected to a sensor electrode of a sensing chip 1000″ is formed on a substrate 3000′.

A sensor electrode pad and a heater electrode pad are connected to the upper part of a first connecting portion of the sensing chip 1000″ and a metal pattern 3100′ is connected to the lower part of a first connecting portion.

The metal pattern 3100′, connected to one of the sensor electrode pads, is connected to one side of a resistor array 400′. And the upper part of a metal portion 3200 is connected to the other side of the resistor array 400′. And the remaining metal pattern 3100′ is directly connected to the metal portion 3200.

The resistor array 400′ is disposed inside a cover 2000. A filter 4000′ is attached in the lower surface of the upper plate of the cover 2000.

### Embodiment 12

In describing the micro sensor package according to the twelfth exemplary embodiment of the present invention, same symbols will be used for the same or similar elements as those of the micro sensor package according to the first, second, third, fourth, fifth, sixth, seventh, eighth, ninth, tenth, and eleventh exemplary embodiments of the present invention, and the detailed description and illustration will be omitted.

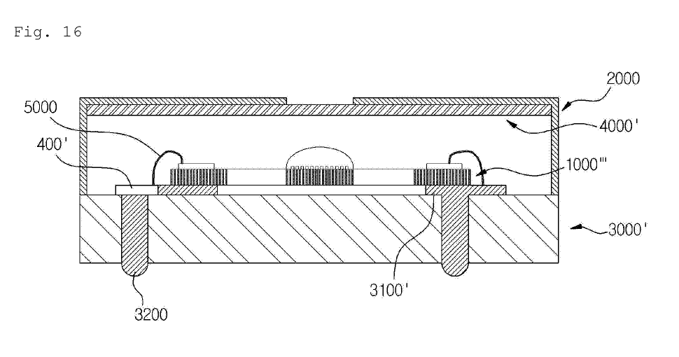

As illustrated in FIG. 16, in a micro sensor package according to the twelfth exemplary embodiment, a sensor electrode of a sensing chip 1000″ and a metal pattern 3100′ formed on the upper surface of a substrate 3000′ are connected to each other through a wire 5000 and a resistor array 400′.

A second sensor electrode pad and a heater electrode pad of the sensing chip 1000″ are connected to the metal pattern 3100′ through the wire 5000. A metal portion 3200 is connected to the metal pattern 3100′.

A first sensor electrode pad is connected to one side of the resistor array 400′ through the wire 5000.

The upper portion of the metal portion 3200 is connected to the other side of the resistor array 400′.

The wire 5000 and the resistor array 400′ are disposed inside a cover 2000. A filter 4000′ is attached in the lower surface of the upper plate of the cover 2000.

According to the micro sensor package of the present invention as described above, the following effects can be obtained.

The thermal conductivity (of the micro sensor package) can be reduced by comprising: a substrate on which a metal pattern is formed; a sensing chip disposed on the substrate; a cover covering the sensing chip and formed with a hole for supplying gas to the sensing chip; and a filter covering the hole, wherein the sensing chip comprises: a sensor platform having a plurality of first pores formed along the up-down direction; and a sensor electrode formed on an upper portion or a lower portion of the sensor platform and electrically connected to the metal pattern, so that it (the micro sensor package) can be maintained at a high temperature with a low power.

A plurality of second pores for supplying gas to the sensing chip is penetratingly formed in the cover along the up-down direction, so that there is an advantage in that a separate filter is not required to be installed.

The substrate is provided with a PCB, so that the micro sensor package can be manufactured at a low cost.

A plurality of third pores is formed in the substrate along the up-down direction, so that the thermal conductivity may be reduced further.

The filter is hydrophobic treated so that the gas sensing portion is prevented from being infiltrated by moisture.

The filter is installed outside the cover so that the filter can be easily installed on the cover

The filter is surface treated in a way that a specific gas is selectively passing through, so that a specific gas can be passing through or blocked, thereby improving the measurement precision.

The resistor array is formed on the same surface as the surface on which the sensor electrode is formed, so that the resistor array can be easily formed and the volume of the micro sensor package can be reduced.

The first pores are penetratingly formed along the up-down direction, and inside of the first pores, a first connecting portion for electrically connecting the sensor electrode and the metal pattern is formed, so that it can be electrically connected without wire bonding.

A plurality of third pores is formed in the substrate along the up-down direction, and a second connecting portion which is electrically connected to the metal pattern is formed inside the third pores, the micro sensor package can be mounted on the printed circuit board without wire bonding.

As described above, although the present invention has been described with reference to the preferred exemplary embodiments, various changes and alterations of the present invention can be made by those skilled in the art without departing from the spirit and the scope of the present invention written in the claims described herein below.

1. A micro sensor package comprising:
a substrate on which a metal pattern is formed;
a sensing chip disposed above the substrate; and
a cover covering the sensing chip,
wherein the sensing chip comprises:
a sensor platform having a plurality of first pores formed along an up-down direction; and
a sensor electrode formed on an upper portion or a lower portion of the sensor platform and electrically connected to the metal pattern,
and wherein in the cover, a plurality of second pores for supplying gas to the sensing chip are penetratingly formed along the up-down direction.

2. The micro sensor package according to claim 1,
wherein the sensor platform is an anodized film obtained by anodizing a base material made of a metal and then removing the base material.

3. The micro sensor package according to claim 1,
wherein the platform is an anodized porous layer wherein the first pores are penetrating along the up-down direction.

4. The micro sensor package according to claim 1,
wherein in the substrate, a plurality of third pores are formed along the up-down direction.

5. The micro sensor package according to claim 1,
wherein the cover is formed of a metallic material.

6. The micro sensor package according to claim 1,
wherein in the filter, a plurality of fourth pores are penetratingly formed along the up-down direction, and the fourth pores communicate with the hole.

7. The micro sensor package according to claim 6,
wherein the fourth pores are formed by anodizing.

8. The micro sensor package according to claim 1,
wherein the filter is subjected to hydrophobic treatment.

9. The micro sensor package according to claim 1,
wherein the filter is installed outside of the cover.

10. The micro sensor package according to claim 1,
wherein the filter is installed inside of the cover.

11. The micro sensor package according to claim 1,
wherein the filter is surface treated so that a specific gas is selectively passing through.

12. The micro sensor package according to claim 1,
wherein the second pores are formed by anodizing.

13. The micro sensor package according to claim 1,
wherein the cover is subjected to hydrophobic treatment.

14. The micro sensor package according to claim 1,
wherein the cover is surface treated so that a specific gas is selectively passing through.

15. The micro sensor package according to claim 1,
wherein the sensor platform is formed with a resistor array electrically connected to the sensor electrode.

16. The micro sensor package according to claim 1,
wherein the first pores are penetratingly formed along the up-down direction, and a first connecting portion for electrically connecting the sensor electrode and the metal pattern is formed inside of at least a part of the first pores.

17. The micro sensor package according to claim 7,
wherein a second connecting portion electrically connected to the metal pattern is formed inside of at least a part of the third pores.

18. The micro sensor package according to claim 7,
wherein the substrate comprises an anodized porous layer.

19. A micro sensor package comprising:
a substrate on which a metal pattern is formed; and
a sensing chip disposed on the substrate,
wherein the sensing chip comprises:
a sensor platform wherein a plurality of first pores are penetratingly formed along an up-down direction;
a sensing electrode formed on an upper portion or a lower portion of the sensor platform and electrically connected to the metal pattern; and
a first connecting portion for electrically connecting the metal pattern and the sensor electrode is formed inside of at least a part of the first pores.

20. A micro sensor package comprising:
a sensor platform wherein a plurality of first pores are formed along an up-down direction;
a sensing chip comprising a sensor electrode formed in the sensor platform; and
a cover that covers the sensor electrode,
wherein at least a part of the plurality of first pores are penetrating through along the up-down direction,
and wherein a first connecting portion electrically connected to the sensor electrode is formed inside of the first pores.
